(19) United States
(12) Patent Application Publication
(10) Pub. No.: US 2011/0293778 A1
(43) Pub. Date: Dec. 1, 2011

(54) NOVEL YEAST STRAIN AND METHODS OF USE THEREOF

(75) Inventor: Raul Cano, San Luis Obispo, CA (US)

(73) Assignee: FOSSIL FUELS BREWING COMPANY, Oakland, CA (US)

(21) Appl. No.: 13/123,118

(22) PCT Filed: Oct. 9, 2009
(86) PCT No.: PCT/US2009/060277

§ 371 Date: Aug. 3, 2011

Related U.S. Application Data
(60) Provisional application No. 61/104,536, filed on Oct. 10, 2008.

(51) Int. Cl. C12N 1/16; C12G 1/00; C12C 11/00; A23C 9/12; A23L 2/02; A23L 2/38; A23L 1/30; A21D 13/00; A23L 1/28; C12G 3/00

(52) U.S. Cl. 426/15; 435/255.1; 426/62; 426/60; 426/11; 426/51; 426/52; 426/531; 426/590; 426/592; 426/599; 426/648; 426/549; 426/580

(57) ABSTRACT

The present disclosure relates to an isolated yeast strain deposited as NRRL Y-50184. The present disclosure also relates generally to methods of manufacturing of products, including a fermented beverage or a fermented food using yeast cell from the isolated yeast strain or a cell culture derived from the strain.

### CROSS-REFERENCE TO RELATED APPLICATIONS

This application claims the benefit of U.S. Patent Application Ser. No. 61/104,536, filed Oct. 10, 2008, the disclosure of which is hereby incorporated by reference in its entirety.

### FIELD

The present disclosure relates generally to an isolated yeast strain deposited as NRRL Y-50184. The present disclosure also relates generally to methods of manufacturing a fermented beverage using the strain deposited as NRRL Y-50184.

### BACKGROUND

Amber is a natural, amorphous, polymeric glass, fossilized resin derived from plants. Remarkably, amber has the capacity to preserve materials that become entrapped in the plant resins from which it originates. For example, scientists have observed the preservation of biological materials in amber inclusions, including cells and cell components (Poinar G. O., Life in Amber, Stanford University Press, Stanford Calif. (1992). Previous work has been conducted with respect to the extraction of ancient DNA from various sources, including amber. However, very little work has been conducted with respect to the recovery of ancient microorganisms from amber. Microorganisms isolated from ancient materials have been attributed to modern microbial contamination.

Over the last century, microbes and microbial by-products have been commercially developed for industrial (e.g., production of fermented beverages), medical (e.g., production of therapeutics and diagnostics) and/or agricultural applications (e.g., production of pesticides). Notably, relatively few species of microorganisms are presently exploited by these applications. Accordingly, there exists a need for new microorganisms that can be employed in these applications, including in modern industrial processes.

### SUMMARY

The present disclosure relates generally to an isolated yeast strain.

The present disclosure provides an isolated yeast strain deposited as NRRL Y-50184 or subcultures thereof.

The present disclosure also provides a biologically pure (e.g., axenic) culture of a yeast strain deposited as NRRL Y-50184 or subcultures thereof. The present disclosure also provides a biologically pure (e.g., axenic) culture of a yeast having all the identifying characteristics of yeast strain deposited as NRRL Y-50184 or subcultures thereof.

In some embodiments, the yeast is in lyophilized form. In some embodiments, the yeast is in powder form. In some embodiments, the yeast is in tablet form. In some embodiments, the yeast is in a capsule.

The present disclosure also provides a yeast cell obtained by a cultivation process of a yeast strain deposited as NRRL Y-50184. The present disclosure also provides a yeast cell that is derived from a yeast strain deposited as NRRL Y-50184. The present disclosure also provides a yeast cell culture comprising yeast cells from the yeast strain deposited as NRRL Y-50184.

The present disclosure also provides a composition comprising a yeast cell from a strain disclosed herein or from a culture disclosed herein and at least one ingredient. In some embodiments, the ingredient is a beverage ingredient. In some embodiments, the ingredient is a food ingredient. In some embodiments, the ingredient is a flavoring ingredient.

The present disclosure also provides for the use of a yeast strain as disclosed herein or a culture as disclosed herein for fermentation.

The present disclosure also provides a method for preparing a yeast cell culture by propagating a yeast cell from a strain as provided herein or from a culture as disclosed herein; and obtaining the yeast cell culture.

The present disclosure also provides a method of preparing a fermented product by contacting a source of sugar with a yeast cell from a strain as disclosed herein or from a culture as disclosed herein; conducting a fermentation process; and obtaining the fermented product. In some embodiments, the fermented product is a beverage. In some embodiments, the beverage is beer, sake, vodka, malt whiskey, wine, cider, brandy, mead, root-beer, ginger-beer, kefir or kumis. In some embodiments, the beer is a lager or an ale. In some embodiments, the fermented product is a food. In some embodiments, the food is a yeast paste, a yeast extract, a probiotic, a food supplement or a bread.

The present disclosure also provides for a fermented product obtained by a method as disclosed herein. The present disclosure also provides for a fermented beverage obtained by a method as disclosed herein. The present disclosure also provides for a fermented food obtained by a method as disclosed herein. The present disclosure also provides for a fermented beverage comprising a yeast cell from a strain as disclosed herein or from a culture as disclosed herein. In some embodiments, the beverage is beer, sake, vodka, malt whiskey, wine, cider, brandy, mead, root-beer, ginger-beer, kefir or kumis. In some embodiments, the beer is a lager or an ale. In some embodiments, the beverage is alcoholic. In some embodiments, the beverage is non-alcoholic. In some embodiments, the product is a food. In some embodiments, the food is a yeast paste, a yeast extract, a probiotic, a food supplement or a bread. In some embodiments, the product is a beverage. In some embodiments, the beverage is beer, sake, vodka, malt whiskey, wine, cider, brandy, mead, root-beer, ginger-beer, kefir or kumis. In some embodiments, the beer is a lager or an ale.

The present disclosure also provides a beer produced by conducting a fermentation process with a yeast cell from a strain as disclosed herein or from a culture as disclosed herein. The present disclosure also provides a yeast cell obtained from a beer as disclosed herein. The present disclosure also provides a yeast cell culture comprising yeast cells obtained from a beer as discloses herein.

The present disclosure also provides methods for preparing a yeast cell culture comprising propagating a yeast cell from a beer as disclosed herein; and obtaining the yeast cell culture.

The present disclosure also provides a method of manufacturing a fermented beverage by conducting a wort production process; and conducting a fermentation process with a yeast strain as disclosed herein. In some embodiments, the method may further comprise conducting a malting process. In some embodiments, the fermented beverage is a beer. In some embodiments, the beer is a lager or an ale. In some embodiments, the methods may further comprise adding hops during the wort production process. In some embodiments, the methods may further comprise a conditioning process.

The present disclosure also provides a kit for preparing a fermented product comprising a yeast cell from a strain as disclosed herein or from a culture as disclosed herein. In some embodiments, the kit is a home brew kit. In some embodiments, the fermented product is a beverage. In some embodiments, the beverage a beer, sake, vodka, malt whiskey, wine, cider, brandy, mead, root-beer, ginger-beer, kefir or kumis. In some embodiments, the beer is a lager or an ale. In some embodiments, the fermented product is a food. In some embodiments, the food is a yeast paste, a yeast extract, a probiotic, a food supplement or a bread.

### DETAILED DESCRIPTION

The present disclosure provides a novel yeast strain designated AY108 and deposited as NRRL Y-50184 (deposited on Oct. 7, 2008 with NRRL, Peoria, Ill.). Recently, methods have been reported to recover and culture ancient organisms obtained from amber (see, e.g., U.S. Pat. No. 5,593,883). Such methods have been employed to recover and isolate a yeast strain from a 45 million-year-old piece of Burmese amber. The isolated yeast strain exhibits carbohydrate consumption properties similar to Saccharomyces and resembles Saccharomyces in both color and texture. This yeast strain may be used in the manufacture of a fermented beverage (e.g. beer). Surprisingly, in the manufacture of beer, the yeast strain exhibits properties that make it amenable to the manufacture of both a lager and an ale (e.g., they settle to the bottom of the wort and they can ferment at a high temperature, such as 20° C.). Additionally, in such manufacture, the yeast strain consumes sugars more rapidly than presently known yeast strains that may result in fermented liquid materials (e.g. beer) with an unusually low specific gravity (e.g., 1.010).

The present disclosure provides methods for manufacturing fermented beverages, including, for example, beer. These methods may comprise a series of processes, including: a malting process, a wort production process and a fermentation process. A malting process is a process in which a grain may be germinated to produce malt. After germination, the malt may be kilned and its root removed. Optionally, the malt may be ground or milled. Alternatively, a malt may be obtained, including purchased from any commercial source, and used directly in a wort production process. In a wort production process, brewing water may be added to the malt (e.g., obtained from a malting process or obtained from a commercial source of malts), thereby producing a mash by permitting enzymes in the malt to convert starch to sugars. In the process of manufacturing fermented beverages (e.g., beers) adjuncts (e.g., rice, starch) may also be added with brewing water. Mash may be lautered and then boiled after hops are added. Such boiling-treatment may be performed to inactivate enzymes in the wort, to make the wort clear by precipitating proteins, to extract and isomerize hop components and/or to sterilize the ingredients. Subsequently, the extract of the wort may be adjusted by the addition of water to the wort after boiling. After cooling of the wort obtained in the wort production process, it may be submitted to the fermentation process. In a fermentation process, a yeast may be added thereby converting sugars in the wort to alcohol. Optionally, a conditioning process may be conducted at the end of the fermentation process to allow the fermented beverage (e.g., beer) to mature.

Styles of beer include that may be manufactured by the methods of the present disclosure include, for example, ales and lagers. Changes in grains, kilning time/temperature, water salt content, hops and yeast strain all contribute to the manufacture of different styles of beer. For example, certain grains or commercially available malts as well as certain hops are known to be useful for the preparation of ales (including various types of ales) whereas other grains or malts as well as other hops are known to be useful for the preparation of lagers (including various types of lagers).

Fermented beverages may include those with alcohol of about 2 to about 15.0 weight %. Preferably, they include those with alcohol of about 4 to about 8 weight %. Adjusting the extract concentration in the wort production process can make final products with the desired concentration of alcohol.

The present disclosure provides methods of fermentation using yeast cells as described herein. Fermentation refers to and includes any process for propagating yeast. The present disclosure provides products of fermentation (e.g., fermented products) including, for example, fermented beverages or fermented foods. Fermented beverages may include, for example, grain-based beverages, fruit-based beverages, honey-based beverages, vegetable-based beverages and dairy-based beverages. Exemplary grain-based beverages may include beer, sake, vodka and malt whiskey. Exemplary fruit-based beverages may include wine, cider and brandy. Exemplary honey-based beverages may include mead. Exemplary vegetable-based beverages may include root beer and ginger beer. Exemplary dairy-based beverages may include kefir and kumis. Fermented foods may include, for example, yeast paste (e.g., nutritional yeast paste), yeast extracts, probiotics, food supplements and breads. Food supplements including as described herein may be used to make beverages (e.g., nutritional beverages).

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Isolation of a Novel Yeast from Amber

Microorganisms (e.g., yeast) may be isolated from amber by known methods in the art. In an exemplary method, a single colony of a yeast was isolated from internal inclusions of leaf and flower parts of a 45 million-year-old piece of Burmese amber by methods as described in U.S. Pat. No. 5,593,883. This colony, designated as AY108, was streaked onto the surface of Sabouraud Glucose agar plate and incubated at 28±0.5° C. for 48 hours to obtain isolated colonies. Resulting colonies were inspected with the aid of a dissecting microscope to assess purity of the culture and 7-8 identical colonies were picked and resuspended in a sterile tube containing 5 milliliters of a sterile solution consisting of 10% glycerol and 1% peptone water. One milliliter aliquots were distributed into CryoTubes™ (Nunc, Inc.) and stored at −70° C. for preservation and long-term storage.

Slide cultures were also prepared from some of the remaining colonies on the plate, stained with lactophenol cotton blue, and examined with the aid of a compound microscope to verify the yeast nature of the isolate and determine its morphological characteristics. The isolated yeast resembled Saccharomyces sp. In both color and texture.

For the preparation of a yeast deposit, a tube (e.g., a Cryo-Tube™, Nunc, Inc.) containing AY108 was removed from the −70° C. freezer and the contents allowed to thaw. The thawed yeast suspension was then used to inoculate several Sabouraud Glucose agar slants and Sabouraud Glucose broth. These tubes were tightly sealed, wrapped with Parafilm™ and shipped to NRRL. The viability of the transferred strain was confirmed by testing at NRRL and the deposit was accorded the NRRL depository number NRRL Y-50184 on Oct. 7, 2008.

### Example 2

### Characterization of a Yeast Isolated from Amber

Yeast isolated from amber may be characterized to assess their carbon assimilation profile and to determine the genus and possibly the species of the isolate.

Isolate AY108 was characterized biochemically using an API ID 32C strip (bioMerieux, France) to assess its carbon assimilation profile. The ID 32C strip consists of 32 cupules, each containing a dehydrated carbohydrate substrate, constituting a miniaturized assimilation system. The analytical profile index for the ID 32C system is based on an eight-digit profile determined with the substrates sorbitol, D-xylose, ribose, glycerol, rhamnose, palatinose, erythritol, melibiose, glucuronate, melezitose, gluconate, levulinate, galactose, actidione, sucrose, N-acetylglucosamine, DL-lactate, L-arabinose, cellobiose, raffinose, maltose, trehalose, 2-ketogluconate, and α-methyl-D-glucoside. Additional tests to be used in cases of low discrimination, for example, when indicated by the analytical profile index, include: sorbose, glucosamine, esculin, mannitol, lactose, and inositol. Glucose may be included as a positive control, and a blank well may be included as a negative control. In an exemplary method, yeast isolate AY108 was grown on Sabouraud Glucose agar at 28° C. A McFarland No. 2 suspension was prepared in sterile physiological saline. One milliliter of this suspension was inoculated into ID 32C medium and 5 drops were dispensed into each well of the strip using an API pipette. The strips were incubated for 24-48 hours at 30±0.5° C. in a sealed container to prevent evaporation. The strips were read to give a carbon assimilation profile. Surprisingly, AY 108 was shown to assimilate glucose, sucrose, DL-lactose, raffinose, and maltose only (Table 1). The eight-digit profile obtained from the results of the ID 32C strip indicated that AY108 resembled Saccharomyces.

TABLE 1
Carbohydrate Utilization Patterns of AY 108
Fermentation Results

| Sugars | Utilization | Sugars | Utilization |
|---|---|---|---|
| Galactose | − | Maltose | + |
| Sorbitol | − | Glucoronate | − |
| Actidione | − | Trehalose | − |
| D-xylose | − | Melezitose | − |
| Sucrose | + | 2-keto-glucose | − |
| Ribose | − | Gluconate | − |
| N-acetyl-glucose | − | α-methyl-D-glucoside | − |
| Glycerol | − | Levulinate | − |
| DL-lactate | + | Mannitol | − |
| Rhamnose | − | Glucose | + |
| L-arabinose | − | Lactose | − |
| Palatinose | − | Sorbose | − |
| Cellobiose | − | Inositol | − |
| Erythritol | − | Glucosamine | − |
| Raffinose | + | Esculin | − |
| Melibiose | − | NEG. CONTROL | − |

### Example 3

### Preparation of a Yeast Isolated from Amber for Use in Manufacture of a Fermented Beverage or Food

A yeast isolated from amber (e.g., AY108, including deposited yeast strain NRRL Y-50184) may be prepared for use in manufacturing a fermented product, including a fermented beverage or fermented food. A variety of growth media and growth conditions may be used to culture the yeast under sterile conditions.

In an exemplary method, a one milliliter CryoTube™ (Nunc, Inc.) containing strain AY108 was partially thawed and a 10 μL aliquot removed using a sterile inoculation loop. Strain AY108 was added to 50 mL of sterile malt extract broth (50 g/L) and incubated at 28° C. for 24 hours. The yeast suspension was transferred to a 5 L flask containing 1 L of malt extract broth and incubated at 28° C. while shaking for 48 hours. The broth was decanted and the remaining yeast sediment (approximately 100 milliliters) was used as inoculum for 11.3 L (approximately 3 gallons) in an 18.9 L (5 gallon) bottle. The yeast was incubated with shaking for 48-72 hours at 28° C. For example, the yeast is grown to concentrations of 10⁸ or greater (e.g., 1-5×10⁸/mL or >5×10⁸/mL, such as 1×10⁹/mL). After the incubation, the yeast was allowed to sediment and the sediment or portion thereof was used as the pitch for the fermentation of a fermented beverage or food product in a tank (e.g., using 8 barrel or 12 barrel fermentation tanks). The yeast sediment or portion thereof is also used as an inoculum (e.g., a starter culture) in methods for the preparation of a yeast-based product, including a fermented beverage or a fermented food.

In another exemplary method, a one milliliter of™ (Nunc, Inc.) containing strain AY108 was partially thawed and a 10 μL aliquot removed using a calibrated, sterile, inoculation loop. Strain AY108 was streaked for isolation onto a plate of malt extract agar (Hardy Diagnostics, catalog number W28) and incubated at 28° C. for 48 hours. After incubation, the plate was examined for the presence of characteristic, isolated colonies of strain AY108. Portions of 2-5 colonies were picked with a sterile inoculating loop and used to inoculate 5 mL of sterile light malt extract broth (Hardy Diagnostics, pale dry malt) with specific gravity of 1.035 in a 15×125 mm tube. The tube was incubated for 24 hours at 28° C. After incubation, the tube was examined for evidence of growth and gas formation. The yeast culture was used to inoculate 50 mL of the sterile light malt extract broth with specific gravity of 1.035 and incubated for 24 hours in an orbital shaker incubator at 28° C. at 200 RPM. The yeast suspension was then transferred to a 5 L flask containing 1 L of the sterile light malt extract broth with specific gravity of 1.035 and incubated for 48 hours in an orbital shaker incubator at 28° C. at 200 RPM. The yeast suspension was used to inoculate 10 L of the sterile light malt extract broth with a specific gravity of 1.035 in an 11.3 L container and incubated for 48 hours in an orbital shaker incubator at 28° C. at 200 RPM. After incubation, the yeast was allowed to sediment and the supernatant fluid siphoned off. An additional 10 L of the sterile light malt extract broth with specific gravity of 1.035 were added to the yeast sediment and incubated with shaking for an additional 48 hours. For example, the yeast is grown to concentrations of 10⁸ or greater (e.g., 1-5×10⁸/ml or >5×10⁸/ml, such as 1×10⁹/ml). After the incubation, the yeast was allowed to sediment and the sediment or a portion thereof was used as the pitch for a fermentation process, for example, using 8 barrel or 12 barrel fermentation tanks. The yeast sediment or portion thereof is also used as an inoculum (e.g., a starter culture) in methods for the preparation of a yeast-based product, such as a fermented product including a fermented beverage or a fermented food.

In another exemplary method, a starter culture (e.g., an active yeast starter) is prepared by streaking an aliquot of a yeast (e.g., a yeast as described in Examples 1-3) onto an agar plate (e.g., malt extract agar, Hardy Diagnostics, catalog number W28) followed by incubation at approximately 28° C. for several hours (e.g., 48 hours). After incubation, portions of a few colonies (e.g., 2-5 colonies) are then picked (e.g., with a sterile inoculating loop) and used to inoculate sterile light malt extract broth (e.g., 5 mL with specific gravity of 1.035) in a tube (e.g., 15×125 mm tube). Alternatively, dried yeast may be used to inoculate the sterile light malt extract broth. Next, the tube is incubated for several hours (e.g., 24 hours) at approximately 28° C. and examined for evidence of growth and gas formation. The yeast culture is then used to inoculate sterile light malt extract broth (e.g., 50 mL with specific gravity of 1.035) and incubated for several hours (e.g., 24 hours) in an orbital shaker incubator (e.g., at approximately 28° C. and 200 RPM). The yeast suspension is then transferred to a flask (e.g., 5 L flask) containing sterile light malt extract broth (e.g., 1 L with specific gravity of 1.035) and incubated for several hours (e.g., 48 hours) in an orbital shaker incubator (e.g., at approximately 28° C. and 200 RPM). After the incubation the yeast is allowed to sediment and the sediment or a portion thereof may be used in methods of making products, including, fermented product such as a fermented beverage or a fermented food. The yeast may be recovered and kept in lyophilized form or in the form of a powder, granule or tablet as well as encapsulated (e.g., in capsules such as hard or soft gelatin capsules) or suspended in liquid media to a desired volume.

### Example 4

### Manufacture of a Fermented Beverage or Food with a Yeast Isolated from Amber

A yeast isolated from amber (e.g., AY108, including deposited yeast strain NRRL Y-50184) may be used in the manufacture of a fermented product, including a fermented beverage (e.g., beer) or a fermented food.

In an exemplary method for the manufacture of an exemplary fermented beverage such as beer, one or more grains (e.g., barley, wheat, rice and/or corn) are steeped in water for a period of time (e.g., 14-18° C. for approximately 48 hours) to raise the grain's moisture content and then allowed to germinate over several days (e.g., 3-5 days). Next, the germination may be stopped by kilning or heating the grain to high temperature (e.g., temperatures below 110° C.) which preserves the amylases and subsequent sugars, thereby producing a malt. The malt may then be ground or milled to fine particles (e.g., by milling). Alternatively, a malt may be obtained from a commercial source and used in the subsequent process steps. Next, the malt (e.g., produced by a malting process or obtained from a commercial source) may be added to water at high temperature (e.g., 65° C.) with well characterized salt profiles (e.g., high calcium is good for ales and low calcium is good for lagers such as pilsners). After approximately 1 hour of heat, the liquid portion (e.g., wort) may be recovered and transferred to a kettle and boiled (e.g., for one hour). This heating may be used to sterilize the wort, precipitate the proteins and remove unpleasant grain flavors. Additionally, during this boil, hops may be added at varying times for bitterness and flavoring. Various hops are commercially available, including hops for beers, ales or lagers. After the boiling, the wort may be cooled, inoculated with a yeast (e.g., a yeast prepared by the method described in Example 1) and aerated to trigger yeast metabolism. Typically, for ales, yeast strains (e.g., Saccharomyces) that collect on the surface of the wort may be used. Typically, for lagers, yeast strains (e.g., Saccharomyces) that settle on the bottom of the wort may be used. Ale fermentation may take a few days at higher temperatures (e.g., approximately 20° C.) and lager fermentation may take several weeks at lower temperatures (e.g., approximately 6° C.). Surprisingly, the yeast strain isolated as described in Example 1 and deposited as NRRL Y-50184 ferments at higher temperatures and yet settles on the bottom of the wort (e.g., is not a surface strain). Such a strain may be useful for the production of ales and lagers. The beer may then be conditioned to settle out the yeast and solids. If desired, additional hops and CO₂ can be added before packaging. For example, an exemplary batch (e.g., 8 barrel batch) of beer (e.g., ale) is prepared as follows. Various malts including, pale malts (e.g., 250 pounds), Munich malts (e.g., 50 pounds) and wheat malts (e.g., 50 pounds) are added into a mash tun containing water (e.g., water at 180° F.) for a period of time (e.g., 10 minutes) and then batch sparged for an additional period of time (e.g., an hour). Next, the wort is pumped into the boil kettle and brought to a boil. Cascade hops are then added at the start of the boil and during the boil (e.g., 30 minutes into the boil). Hallertau hops are additionally added during the boil (e.g., 30 minutes, at 60 minutes, and the end of the boil). The wort (e.g., at a specific gravity of 1.048) is cooled (e.g., to 68° F.) and AY108 yeast (e.g., a pitch prepared as described in Example 3) is added. Next, the wort is sparged with oxygen, stirred and the fermentation tank sealed. The initial yeast growth is rapid with violent top fermentation, the temperature range is narrow and warm (e.g., between 20-21° C.) and is preferably kept in this range for active fermentation. In addition, the yeast flocculates quickly, even with high residual sugar levels and is preferably occasionally agitated and re-circulated during the entire fermentation. In this exemplary 8-barrel batch, the sugar attenuation rate is high (e.g., approximately 80%) and an ale beer is produced with a final specific gravity of 1.014. The beer is then drawn off to a finishing tank and held for several weeks at a temperature (e.g., two weeks at 2-8° C.) and served unfiltered. For another example, an exemplary batch (e.g., 10 barrel batch) of beer (e.g., wheat) is prepared. The process is similar to the process for the ale beer described above but using Pilsner malt (e.g., 610 pounds), White Wheat malt (e.g., 750 pounds), and Sterling hops and step mash (e.g., 145° F. for 15 minutes raised to 155° F. over 15 minutes, then mashed at 155° F. for 20 minutes and with the mash out at 168° F.). The fermentation observations are also similar. A wheat beer is produced with a final specific gravity of 1.010.

In an exemplary method for the manufacture of another exemplary beverage such as sake (e.g., a beverage containing approximately 18-20% ethanol), rice is used in a fermentation process similar to that of beer making, including as described above. Grains, such as rice (e.g., California Pearl, Mochi and Gomi), are milled to remove the hulls, bran and aleurone layers, leaving the starch-rich endosperm consisting of broken kernels. Next, the kernels are ground to particles (e.g., <2 mm) and mashed at 36° C.-42° C. for a period of time (e.g., variable time) after the addition of malt grist (e.g., amylase and protease source) to liquefy the mash. The mash is then boiled for a period of time (e.g., 15 minutes) to gelatinize the mash and to stop the starch reduction. Next, a yeast starter culture (e.g., a yeast as described in Examples 1-3) is added to the mash with ammonium salts to initiate fermentation. The production of sake from rice involves a single fermentation step at slightly higher temperatures than beer using alcohol tolerant yeast.

In an exemplary method for the manufacture of another exemplary beverage such as vodka, sugars from grain (e.g., rye or wheat), potatoes, or sugar beet molasses are fermented with yeast (e.g., a yeast as described in Examples 1-3). The grain (e.g., in the form of wheat) or vegetables are placed inside a mash tub that rotates to break down the grains. Next, malt grist is added to the mash for the conversion of the starch to sugars. The mash is then heated until it reaches boiling point to sterilize the mash, followed by the addition of acid bacteria to increase the acidity level needed for the vodka fermentation process. The mash is again sterilized by boiling. Next, the mash is then streamed into tanks (e.g., stainless steel tanks), a yeast starter culture is added (e.g., a yeast as described in Examples 1-3) and the mash is fermented for a few days (e.g., four days). The ethanol is then distilled using a still (e.g., a column still comprised of vaporization chambers stacked on top of each other or pot still). The alcohol is continuously heated (e.g., with steam) in the still while it cycles up and down in the column. This cycle continues until the vapors created from the heat are released and condense at the top vaporization chambers. The by-products and extracted materials drain into the lower chambers where they can be discarded. The vapors created by the distillation process (e.g., fine spirits), contain between approximately 95%-100% alcohol. The vapors may be made suitable for drinking by adding water and flavorings to the vapors to dilute the alcohol concentration from approximately 100% to approximately 40%.

In an exemplary method for the manufacture of another exemplary beverage such as malt whiskey, a grain (e.g., barley or rye) is dampened and allowed to germinate, which changes the starch contained in the grain to sugar to form a malt. The end of the germination is triggered by drying the germinating grain over a fire (e.g., a kiln). When the malt is dry, it is ground to a coarse flour called grist. Next, the grist is mixed (e.g., 1:4 volume of grist to water) with hot water (e.g., at a temperature between approximately 63-95° C.) in a mash tun to extract the sugars, for example, with three successive waters. The sugared water is called wort. Yeast (e.g., a yeast as described in Examples 1-3), for example, a yeast starter culture is then added and the wort fermented (e.g., similar to beer fermentation) to an alcohol percentage of approximately 8%. The fermented product is then distilled to separate the alcohol from water and other substances contained in the fermented wort. The quality of the distillation depends partially on the type of surface (e.g., copper surface) in contact with the liquids during the distillation process and other still features (e.g., the shape, the height, the length of the lyne arm). Distinctive flavors may be generated during the aging process where several factors (e.g., the casks used, the nature of the warehouse, the taste of the air) may exert an effect. For example, storage in casks and aging for up to 15-20 years may be used to impart characteristic flavors and aromas.

In an exemplary method for the manufacture of another exemplary beverage such as wine, crushed grapes are fermented using yeast (e.g., yeast as described in Examples 1-3), which ferments sugars found in the grapes and converts them into alcohol. Grapes are harvested, for example, during the cool morning hours, when the sugar content is at its peak and moved to the winery in open bins. Next, the grapes are transferred to a stemmer/crusher where the stems are removed and the grapes are crushed. After crushing and de-stemming, the juice, now called must, is placed into the fermentation vats and a yeast starter culture is added to start the fermentation. The primary fermentation therefore takes place with free access to the air at a temperature for several days (e.g., red wines usually at 70-90° F. for 5-10 days and whites at 55-80° F. for 10-15 days. The young wine is run off into casks or vats which are covered from the air for the secondary fermentation. After this fermentation, the wine is transferred (e.g., racked) into a different vessel. Next, the wine is aged in tanks (e.g., stainless steel tanks) or barrels (e.g., oak barrels) depending upon the style of wine being fermented. After aging and prior to bottling, the wines are fined and filtered to stabilize and clarify them. Next, wines are bottled in a sterile environment, and sealed (e.g., with a cork). Different varieties of grapes and the presence or absence of skin produce various types of wine.

In an exemplary method for the manufacture of another exemplary beverage such as cider, apple juice is fermented by yeast (e.g., yeast as described in Examples 1-3). In this process, the apples (e.g., approximately 1000 pounds) are first washed, then inspected to make sure that they are clean and whole. Next they are fed into a grinding mill that mashes them into a texture resembling that of applesauce but with seeds and skin included. Next, the pulp is pumped or scooped for filtration onto filters. For example, the filters may be a woven cheesecloth placed in a square frame above a wooden rack such as an open checkerboard of thin oak slats crosswise to each other. After each cloth is filled with pulp (e.g., about five gallons), it is folded over and another rack and cloth are placed on top. In addition, for example, a dozen or so racks and cloths are filled and stacked into a pile (e.g., about three feet high)and rolled into a viselike press (e.g., that applies a pressure of 2,000 to 3,000 pounds per square inch to the whole stack). Following filtration, the apple juice in poured into casks or other fermentation vessels. Next, a yeast (e.g., a yeast as described in Examples 1-3), for example, a yeast starter culture is added and the apple juice is allowed to ferment (e.g., at a temperature of 4-16° C.). Shortly before the fermentation consumes the sugar, the liquor is racked (e.g., siphoned) into new vats, leaving dead yeast cells and other undesirable material at the bottom of the old vat. Finally, vats are filled completely to exclude air including, to exclude airborne acetic bacteria, and the fermentation is allowed to continue. The remaining available sugar generates a small amount of carbon dioxide that forms a protective layer, reducing air contact. This final fermentation also creates a small amount of carbonation. Extra sugar may be added specifically for this purpose. Racking is sometimes repeated if the liquor remains too cloudy. The cider is ready to drink after a fermentation period (e.g., three month fermentation period), though more often it is matured in the vats for several years (e.g., up to two or three years).

In an exemplary method for the manufacture of another exemplary beverage such as brandy, yeast (e.g., yeast as described in Examples 1-3) are used in the fermentation of a liquid that contains any form of sugar. Sources of sugar may include, for example, grapes or other fruits (e.g., grapes, apples, blackberries), vegetables (e.g., potato), sugar cane, honey, milk, rice, wheat, corn, or rye. French brandies are made from the wine of the St. Emillion, Colombard (e.g., Folle Blanche) grapes. In an exemplary method, brandy may be produced by the alcoholic fermentation of grapes (e.g., white wine grapes) in a process similar to that of wine making. The grapes are allowed to ferment to an alcohol content of approximately 10%. After fermentation the wine is distilled to purify and increase the alcohol concentration to approximately 36-60%. Brandy is usually double-distilled, meaning that the alcohol is concentrated twice. For example, it takes about 9 gallons (34 L) of wine to make 1 gallon (3.8 L) of brandy. After a first distillation, which takes about eight hours, the concentrated liquid has an alcohol content of approximately 26-32%. The product of a second distillation has an alcohol content of approximately 72%. The higher the alcohol content the more neutral (e.g., tasteless) the brandy. Next, the brandy is transferred to oak casks and allowed to age. Most brandy consumed today, even fine brandy, is less than six years old. However, some fine brandies are more than 50 years old. As the brandy ages, it absorbs flavors from the oak while its own structure softens, becoming less astringent. Through evaporation, brandy will lose about 1% of is alcohol content per year for the first 50 years or so that it is on oak.

In an exemplary method for the manufacture of another exemplary beverage such as mead, honey and water is fermented with a yeast (e.g., a yeast as described in Examples 1-3). A must is formed by diluting the honey in water (e.g., 1:4-1:66), adding ammonium salts and growth factors, and steeping for a suitable time and temperature (e.g., 15 minutes at 65° C.). After cooling (e.g., to 21° C.), a yeast (e.g., a yeast as described in Examples 1-3), for example, a yeast starter culture is added and the primary fermentation is allowed to take place for several days (e.g., 10-15 days). Next, the must is racked to a clean fermentor, additional growth supplements added and the temperature reduced (e.g., to 18° C.). The must is allowed to stay at this temperature for a period of days (e.g., 120 days), racked into a third fermentation vessel and held at lower temperatures (e.g., temperatures ranging from approximately 2-16° C.) for up to a couple of years (e.g., two years), depending on the desired taste. The alcohol concentration of the final product varies (e.g., from approximately 5 to 14%) and may have a slight carbonation depending of the fermentation conditions. Mead may be flavored with hops and/or spices.

In another exemplary method for the manufacture of another exemplary fermented beverage such as root beer, sassafras roots (e.g., 50 g) and hops (e.g., 28 grams) is steeped in boiling water (e.g., 7.5 L) for a period of time (e.g., 20 minutes). The root extract is allowed to cool to at approximately 28-30° C. and then strained (e.g., through several layers of cheese cloth) into a sterile, container with cap (e.g., 19 L). Alternatively, root beer extracts may be obtained from commercial sources such as Shank's Extracts (Lancaster, Pa.) or Zatarain (New Orleans, La.). Next, sugar (e.g., approximately 2.3 kg) is added and mixed well until it is completely dissolved. When the sugar is dissolved, a yeast (e.g., a yeast as described in Examples 1-3), for example, a yeast starter culture is added to a final concentration of ≧1×10⁸ cells/mL. After the yeast is added, the container is sealed tightly (e.g., with a cork or plastic cap), then stored for a period of time (e.g., 6-8 hours) in a warm place then refrigerated for a period of time (e.g., 24 hours). Alternatively, the sweetened root beer extract and the yeast can be bottled (e.g., in 24 ounce bottles), capped and stored.

In another exemplary method for the manufacture of another exemplary fermented beverage such as ginger beer, raw honey (e.g., 15 mL) is added to warm water (e.g., 1 L) in a sterile fermentation vessel (e.g., 3 L). Once the honey is dissolved, a yeast (e.g., a yeast as described in Examples 1-3), for example, a yeast starter culture is added to a final concentration of ≧3×10⁸ yeast cells/mL. The container is then capped and the vessel is allowed to stand in a warm place for a period of time (e.g., approximately 12 hours or overnight). Next, fresh ginger (e.g., 150 g) is peeled and grated. The juice is then squeezed into a disinfected, fermentation vessel (e.g., 3 L) containing water (e.g., 1 L) and honey (e.g., 15 mL). The remaining ginger pulp is steeped by simmering in water (e.g., 250 mL) for a period of time (e.g., 30 minutes). The steeped liquid is allowed to cool and then filtered (e.g., through several layers of gauze) into the fermentation vessel and the remaining pulp steeped as described above. Alternatively, ginger beer extracts may be obtained from commercial sources (e.g., Northern Brewer, Roseville, MN, Home Brew Mart). Next, lemon or lime juice (e.g., 30 mL) is added to the fermentation vessel and sufficient water is added to fill the vessel (e.g., to bring the total volume in the vessel to 3 L). After the juice is added, the container is sealed tightly (e.g., with a cork or plastic cap), and then stored for a period of time (e.g., 5 days). Next, rice syrup (e.g., 10 mL) is dissolved in water (e.g., 100 mL) and added to the fermented ginger beer. The ginger beer is then bottled, capped, and stored with refrigeration until used.

In another exemplary method for the manufacture of another exemplary fermented beverage such as kefir, milk is fermented by yeast and lactic acid bacteria in a matrix of proteins, lipids and sugars (e.g., kefir grains). The milk is obtained from cows, goats or sheep. An aliquot (e.g., 1 L) of cow's milk is mixed with Kefir granules (e.g., 10 mL) containing the yeast (e.g., a yeast as described in Examples 1-3, for example, a yeast starter culture) and other starter microorganisms in a container (e.g., 2 L glass container). The container containing the mixture is then loosely covered (e.g., with plastic wrap or lid) and stored at room temperature for a period of time (e.g., 24 hours). The fermented milk is then filtered (e.g., through several layers of gauze) to remove the kefir grains and stored with refrigeration (e.g., 5° C.) until used. The Kefir grains are stored also in a closed container and can be used as starters for the next batch. The Kefir beverage may be slightly carbonated with a low ethanol content (e.g., 1-2%), depending on the length of the fermentation.

In another exemplary method for the manufacture of another exemplary fermented beverage such as kumis, mare's milk is fermented by a starter culture of yeast (e.g., a yeast as described in Example 1) and clabber. Mare's milk (e.g., 1 L) is heated to a boil with water (e.g., 250 mL) and sugar (e.g., 5 g). Next, the mixture is allowed to cool to room temperature and then mixed with clabber (e.g., 25 mL). The mixture is allowed to incubate, for example, at room temperature, until it sours (e.g., breaks up into cured and whey). Next, a suspension of the yeast (e.g., a yeast as described in Examples 1-3), for example, a yeast starter culture is added to a final concentration of ≧1×10⁸ yeast cells/ml and then incubated for a period of time (e.g., 24-48 hours) until fermentation is completed. The final product is then strained into suitable containers. The kumis beverage appears as a milk product with carbonation and a low alcohol concentration (e.g., 0.5×1.0% ethanol).

In an exemplary method for the manufacture of an exemplary fermented food such as yeast paste (e.g., nutritional yeast paste) or yeast extract (e.g., a food additive or flavoring), a residual yeast slurry resulting from a fermentation using a yeast (e.g., a yeast as described in Examples 1-3), for example, a yeast starter culture (e.g., a 7 barrel fermentation) can be collected from the fermentation vessel and stored in a cool place for several days to promote autolysis. Alternatively, the yeast may be hydrolyzed. Autolysis, or self digestion, refers to the destruction of a cell as a result of the hydrolytic action of its own enzymes. Next, the autolyzed or hydrolyzed yeast is pressed to remove excess liquids to produce a yeast paste (e.g., a nutritional yeast paste) or a yeast extract. The yeast paste is then packaged and may be used as a spread, food additive, or flavor enhancer. Alternatively, the yeast paste can be further flavored with spices and/or artificial flavorings and then packaged. The yeast extract may be used as a food additive or a flavoring agent (e.g., flavoring enhancer).

In another exemplary method for the manufacture of another exemplary food product, such as a probiotic or a food supplement, yeast (e.g., as described in Examples 1-3) can be manufactured including, in tablets, feed pellets or capsules of pressed yeast cells. A yeast starter culture (e.g., an active yeast starter) is prepared, for example, as described in Examples 3. A yeast suspension obtained as described in Example 3 is used to inoculate sterile light malt extract broth (e.g., 10 L with specific gravity of 1.035) in an container (e.g., 11.3 L) and incubated with agitation for several hours (e.g., 48 hours in an orbital shaker incubator at approximately 28° C. and 200 RPM). After incubation, the yeast is allowed to sediment and the supernatant fluid is siphoned off. Additional sterile light malt extract broth (e.g., 10 L with specific gravity of 1.035) is added to the yeast sediment and incubated with agitation for several hours (e.g., 48 hours). For example, the yeast is grown to concentrations of ≧10⁸ (e.g., 1-5×10⁸/mL or >5×10⁸/mL such as 1×10⁹/ml). After incubation, the yeast is allowed to sediment and recovered. The yeast mass is then dried and may be pressed into tablets or encapsulated in capsules containing a desired amount of yeast per tablet or capsule (e.g., approximately 1-10 mg of yeast per tablet).

In another exemplary method for the manufacture of another exemplary food, such as bread is made by baking leavened dough, containing, yeast (e.g., yeast as described in Examples 1-3), water, salt and flour. Yeast participates in the leavening process by fermenting the carbohydrates in the flour to produce carbon dioxide. During bread making, the dough is allowed to rise in a warm place then baked. The rising of the dough is a result of the carbon dioxide production by the yeast during fermentation. Yeasts may also impart characteristic flavors and aromas to the bread. In an exemplary method for the manufacture of exemplary leavened bread and in preparation for the baking process a culture of active yeast starter is prepared for the leavening process. A yeast starter culture is dispended into a mixing bowl along with warm water (e.g., 500 mL) and flour (e.g., 3 cups of whole wheat flour) and salt (e.g., 2 teaspoons). This mixture is mixed will until all of the ingredients are combined. Then additional flour (e.g., 1.5 cups) is added in aliquots (e.g., 0.5-cup), mixing well after each addition. When the dough has pulled together, it is turned out onto a floured surface (e.g., lightly floured surface) and kneaded until smooth and elastic. Next, the resulting dough is placed into a large, lightly oiled bowl and turned to coat with oil. The dough is covered (e.g., with a damp cloth) and allowed to rise in a warm place until it has doubled in volume. The dough is then deflated, turned out onto a lightly floured surface, then divided into two equal loaves. Next, the loaves are placed into lightly-oiled pan (e.g., 9×5 inch loaf pans). The loaves are then covered (e.g., with a damp cloth) and allowed to rise until doubled in volume. Next, the bread dough is baked (e.g., at 220° C.) for a period of time (e.g., 30 minutes or until the top is golden brown) and the bottom of the loaf sounds hollow when tapped.

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. An isolated yeast strain deposited as NRRL Y-50184 or subcultures thereof.

2. (canceled)

3. A biologically pure culture of a yeast strain deposited as NRRL Y-50184 or subcultures thereof.

4. A biologically pure culture of a yeast having all the identifying characteristics of yeast strain deposited as NRRL Y-50184 or subcultures thereof.

5-8. (canceled)

9. A yeast cell obtained by a cultivation process of a yeast strain deposited as NRRL Y-50184.

10. (canceled)

11. A yeast cell culture comprising yeast cells from the yeast strain deposited as NRRL Y-50184.

12. A composition comprising a yeast cell from the culture of claim 11 and at least one ingredient.

13. The composition of claim 12, wherein the ingredient is a beverage ingredient.

14. The composition of claim 12, wherein the ingredient is a food ingredient.

15-17. (canceled)

18. A method of preparing a fermented product, said method comprising
(a) contacting a source of sugar with a yeast cell from the culture of claim 11;
(b) conducting a fermentation process; and
(c) obtaining the fermented product from step (b).

19. The method of claim 18, wherein the fermented product is a beverage.

20. The method of claim 19, wherein the beverage is beer, sake, wine, cider, mead, root-beer, ginger-beer, kefir or kumis.

21. (canceled)

22. The method of claim 18, wherein the fermented product is a food.

23. (canceled)

24. A fermented product obtained by the method of claim 18.

25. A fermented beverage obtained by the method of claim 19.

26. A fermented food obtained by the method of claim 22.

27. A fermented beverage comprising a yeast cell from the culture of claim 11.

28. The fermented beverage of claim 27, wherein the beverage is beer, sake, wine, cider, mead, root-beer, ginger-beer, kefir or kumis.

29-31. (canceled)

32. The fermented product of claim 24, wherein the product is a food.

33. The fermented product of claim 32, wherein the food is a food supplement or a bread.

34-36. (canceled)

37. A beer produced by conducting a fermentation process with a yeast cell from the culture of claim 11.

38. A yeast cell obtained from the beer of claim 37.

39. A yeast cell culture comprising yeast cells obtained from the beer of claim 37.

40-53. (canceled)
